(19) United States
(12) Patent Application Publication
(10) Pub. No.: US 2016/0143597 A1
(43) Pub. Date: May 26, 2016

(54) PROCESSING DEVICE

(71) Applicant: Winbond Electronics Corp., Taichung City (TW)

(72) Inventors: Chia-Chi CHANG, Taipei City (TW); Tzu-Chien HSIAO, Zhubei City (TW)

(21) Appl. No.: 14/552,148

(22) Filed: Nov. 24, 2014

(51) Int. Cl. A61B 5/00; A61B 5/0488; A61B 7/04; A61B 5/0402; A61B 5/0205; A61B 5/0476

(52) U.S. Cl. CPC A61B 5/7282; A61B 5/0205; A61B 5/0476; A61B 7/04; A61B 5/0402; A61B 5/0488; A61B 5/742; A61B 5/0002; A61B 5/746; A61B 5/02416

(57) ABSTRACT

A processing device including an interface unit, a first slot, and a transmission unit is provided. The interface unit is configured to receive a plurality of physiological monitoring signals. The first slot is configured to hold a first analysis module. When the first analysis module is inserted into the first slot, the first analysis module analyzes a first signal group to generate a first analysis result. The first signal group includes at least two of the physiological monitoring signals. The transmission unit outputs the first analysis result to at least one external device.

### BACKGROUND OF THE INVENTION

1. Field of the Invention

The invention relates to a processing device, and more particularly to a processing device for physiological symptoms.

2. Description of the Related Art

Physiological monitors are common medical apparatuses to measure physiological parameters. Doctors and nurses diagnose and treat sick people according to measured physiological indexes. However, different physiological indexes (e.g. electrocardiogram (ECG), blood pressure, temperature and respiration) are captured by different physiological monitors. The cost of monitoring is increased and the complexity of measurement is increased.

### BRIEF SUMMARY OF THE INVENTION

In accordance with an embodiment, a processing device comprises an interface unit, a first slot, and a transmission unit. The interface unit is configured to receive a plurality of physiological monitoring signals. The first slot is configured to hold a first analysis module. When the first analysis module is inserted into the first slot, the first analysis module analyzes a first signal group to generate a first analysis result. The first signal group comprises at least two of the physiological monitoring signals. The transmission unit outputs the first analysis result to at least one external device.

A detailed description is given in the following embodiments with reference to the accompanying drawings.

### BRIEF DESCRIPTION OF THE DRAWINGS

The invention can be more fully understood by referring to the following detailed description and examples with references made to the accompanying drawings, wherein:

FIG. 1 is an appearance diagram of an exemplary embodiment of a processing device according to an embodiment of the present invention;

FIG. 2 is a schematic diagram of an exemplary embodiment of a processing device according to an embodiment of the present invention;

FIG. 3 is a schematic diagram of an exemplary embodiment of an analysis module according to an embodiment of the present invention;

FIG. 4 is a schematic diagram of another exemplary embodiment of a processing device according to an embodiment of the present invention; and

FIG. 5 is a schematic diagram of another exemplary embodiment of a processing device according to an embodiment of the present invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

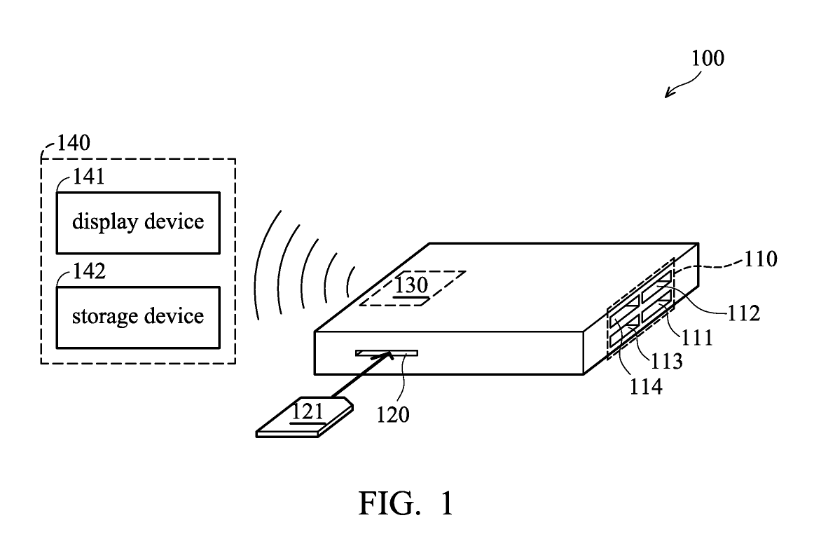

FIG. 1 is an appearance diagram of an exemplary embodiment of a processing device according to an embodiment of the present invention. The processing device 100 comprises an interface unit 110, a slot 120 and a transmission unit 130. The interface unit 110 comprises jacks 111-114. The invention does not limit the number of jacks nor the shape of each jack. In this embodiment, each jack is coupled to a corresponding monitoring device (not shown) to receive at least one physiological monitoring signal. In one embodiment, a monitoring device may generate various physiological monitoring signals.

The monitoring device detects a physiological symptom, such as an electroencephalography (EEG) signal, a phonocardiograph (PCG) signal, an impedance cardiogram (ICG) signal, a photoplethysmogram (PPG) signal, a blood pressure signal, an electrocardiogram (ECG) signal, a respiration signal, an electromyography (EMG) signal and a heart rate signal. The monitoring device provides physiological monitoring signals to the interface unit 110.

The slot 120 is configured to hold an analysis module 121. The invention does not limit the size of the analysis module 121. In one embodiment, the shape of the analysis module 121 is similar to a secure digital card. When the analysis module 121 is inserted into the slot 120, the analysis module 121 analyzes the physiological monitoring signals received by at least two jacks to generate an analysis result.

Different analysis modules analyze different physiological monitoring signals. For example, if the analysis module 121 is a cardiopulmonary function analysis module, the analysis module 121 analyzes the physiological monitoring signals received by the jacks 111 and 112 to generate an analysis result. The physiological monitoring signals received by the jacks 111 and 112 may comprise an ECG signal and a respiration signal. If the analysis module 121 is a blood pressure modulating module, the analysis module 121 analyzes the physiological monitoring signals received by the jacks 112 and 113 to generate another analysis result. The physiological monitoring signals received by the jacks 112 and 113 may comprise a ECG signal and a blood pressure signal. In this case, the same physiological monitoring signal may be utilized by different analysis modules. In other embodiments, different analysis modules utilize different physiological monitoring signals.

The transmission unit 130 transmits the analysis result to an external device 140 according to a wireless transmission technology or a wired transmission technology. The invention does not limit the type of external device 140. In one embodiment, the external device 140 comprises a display device 141. The display device 141 displays a text or an image representing the analysis result generated by the analysis module 121. In one embodiment, the display device 141 is a smart phone or a monitor.

In other embodiments, the external device 140 further comprises a storage device 142 to store the analysis result generated by the analysis module 121. For example, the storage device 142 is a smart phone, a host, or a server. In some embodiments, the transmission unit 130 communicates with the display device 141 according to a wired transmission technology and communicates with the storage device 142 according to a wireless transmission technology. In some embodiments, the transmission unit 130 transmits data to a cloud via the Internet.

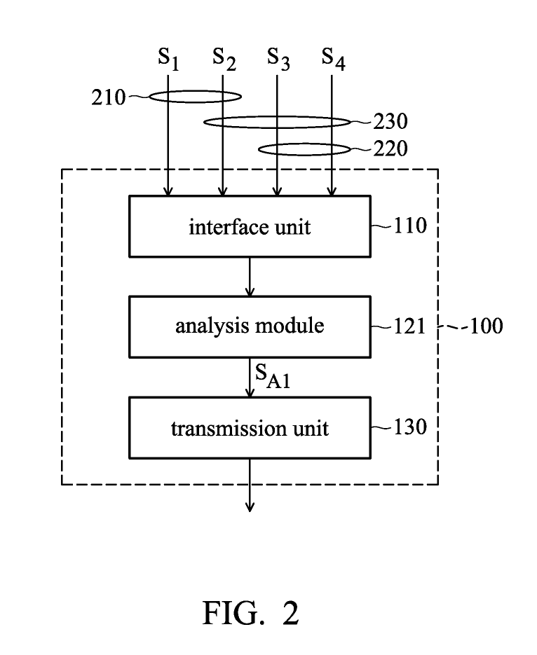

FIG. 2 is a schematic diagram of an exemplary embodiment of a processing device according to an embodiment of the present invention. In order to clearly interpret this embodiment, only physiological monitoring signals S₁˜S₄ are shown. The analysis module 121 analyzes at least two of the physiological monitoring signals S₁˜S₄ to generate an analysis result S_{A1}.

For example, when the analysis module 121 is a first category of analysis module (e.g. a cardiopulmonary function analysis module), the analysis module 121 analyzes the signal group 210. The signal group 210 comprises the physiological monitoring signals S₁ and S₂. When the analysis module 121 is a second category of analysis module (e.g. a blood pressure analysis module), the analysis module 121 analyzes the signal group 220. The signal group 220 comprises the physiological monitoring signals S₃ and S₄. When the analysis module 121 is a third category of analysis module, the analysis module 121 analyzes the signal group 230. The signal group 230 comprises the physiological monitoring signals S₂˜S₄.

Different kinds of analysis modules generate different analysis results. The user obtains the physiological symptom of a patient according to the analysis result. Since the processing device 100 comprises a slot 120, a user is capable of inserting the appropriate analysis module into the slot 120, and the utility of the processing device is increased.

In other embodiments, the analysis module 121 controls the transmission unit 130 to selectively transmit the analysis result S_{A1} to an appropriate external device. For example, if the analysis module 121 is to a first category of analysis modules, the analysis module 121 directs the transmission unit 130 to transmit the analysis result S_{A1} to a first external device, and if the analysis module 121 is to a second category of analysis modules, the analysis module 121 directs the transmission unit 130 to transmit the analysis result S_{A1} to a second external device.

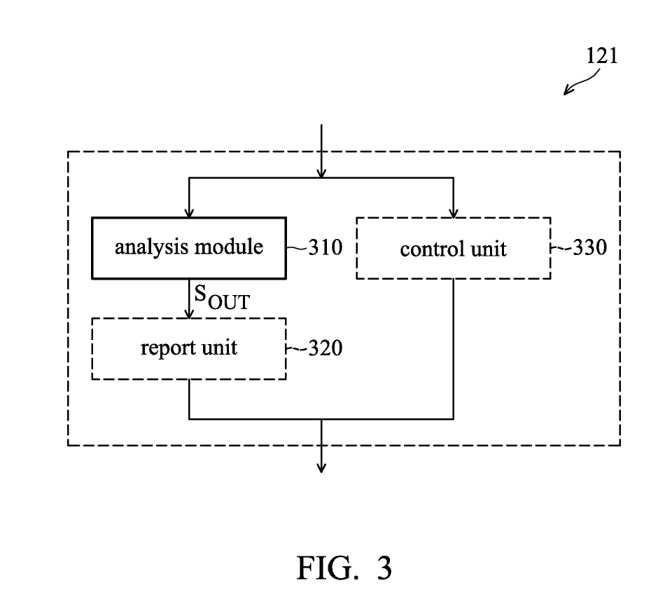

The invention does not limit the internal structure of analysis module 121. Any circuit structure can serve as an analysis module 121, as long as the circuit structure is capable of analyzing physiological monitoring signals. FIG. 3 is a schematic diagram of an exemplary embodiment of an analysis module according to an embodiment of the present invention. In one embodiment, the analysis module 121 comprises an analysis unit 310. The analysis unit 310 analyzes at least two of the physiological monitoring signals S₁˜S₄ to generate an output signal S_{OUT}. In this case, the transmission unit 130 directly receives and transmits the output signal S_{OUT} to the external device.

In another embodiment, the analysis module 121 further comprises a report unit 320. The report unit 320 collects the output signal S_{OUT} to generate a report result S_{A1}. For example, the report unit 320 outputs an analysis result S_{A1} at fixed time intervals. Therefore, an user can detect changes in the physiological symptoms of a patient according to the analysis result displayed by the external device.

In other embodiments, the report unit 320 is disposed in an external device to report the analysis result S_{A1} output from the transmission unit 130. In one embodiment, the external device displays text or an image to represent the analysis result S_{A1}. In another embodiment, the external device stores the analysis result S_{A1}.

In some embodiments, the analysis module 121 further comprises a control unit 330 to detect some sudden conditions. When the states of at least two of the physiological monitoring signals S₁˜S₄ conform to a dangerous state, the control unit 330 sends an alarm signal. For example, during analysis period, if the cardiopulmonary function of a patient is abnormal, the control unit 330 immediately sends an alarm signal to notify medical personnel. In other embodiments, the control unit 330 can be disposed outside of the external device.

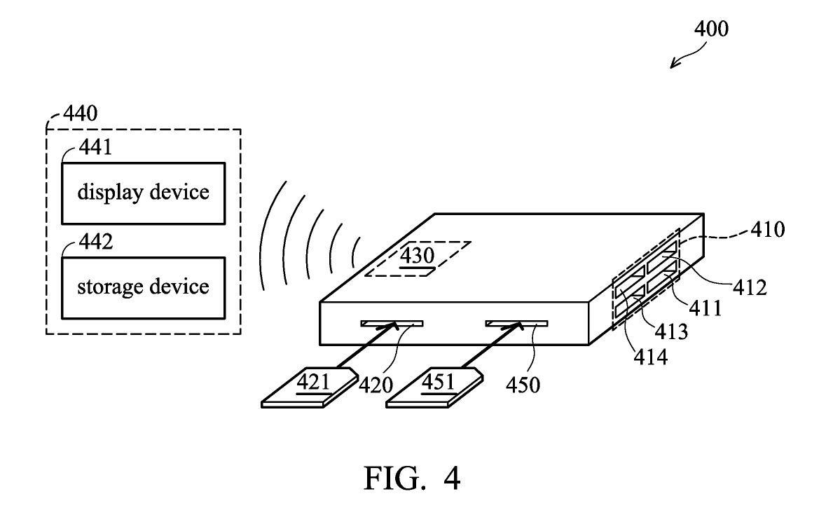

FIG. 4 is a schematic diagram of another exemplary embodiment of a processing device according to an embodiment of the present invention. FIG. 4 is similar to FIG. 1 except for the addition of a slot 450. The slot 450 is configured to hold an analysis module 451. In one embodiment, the analysis modules 421 and 451 can be simultaneously inserted into the slots 420 and 450 respectively. The transmission unit 430 successively provides the analysis results generated by the analysis modules 421 and 451 to the external device 440.

In one embodiment, the transmission unit 430 provides different analysis results to the same external device, such as the display device 441. In another embodiment, the transmission unit 430 provides different analysis results to different external devices. For example, the transmission unit 430 provides the analysis result generated by the analysis module 421 to the display device 441 and provides the analysis result generated by the analysis module 451 to the storage device 442. In other embodiments, the transmission unit 430 transmits data to an external device according to a wireless transmission technology and transmits data to another external device according to a wired transmission technology.

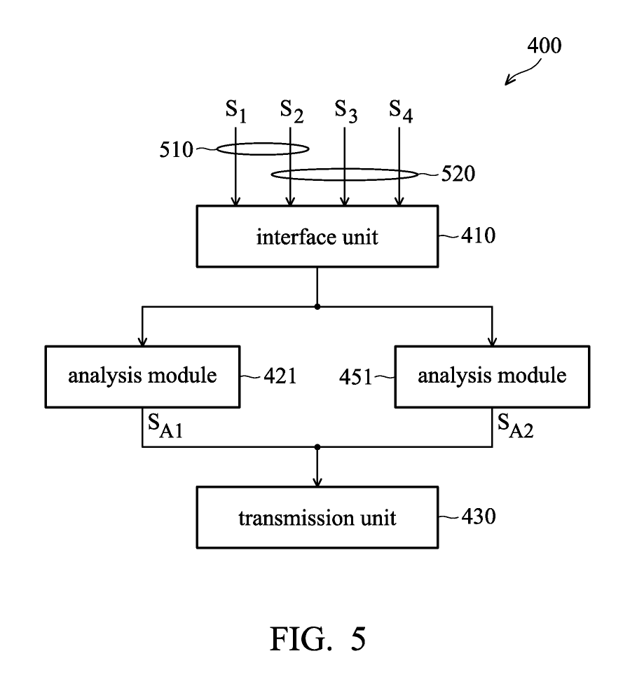

FIG. 5 is a schematic diagram of another exemplary embodiment of a processing device according to an embodiment of the present invention. The physiological monitoring signals S₁ and S₂ constitute the signal group 510 and the physiological monitoring signals S₂˜S₄ constitute the signal group 520. In this embodiment, each of the analysis modules 421 and 451 utilizes the physiological monitoring signal S₂, but the disclosure is not limited thereto. In other embodiments, the physiological monitoring signals analyzed by the analysis module 421 are different from the physiological monitoring signals analyzed by the analysis module 451.

The analysis module 421 analyzes the signal group 510 to generate the analysis result S_{A1}. The analysis module 451 analyzes the signal group 520 to generate the analysis result S_{A2}. The invention does not limit the sequence for which the analysis modules 421 and 451 analyze the physiological monitoring signals. In one embodiment, the priority of the slot 420 is higher than the priority of the slot 450. Therefore, when the analysis modules 421 and 451 are inserted into the slots 420 and 450 respectively, the analysis module 421 first analyzes the physiological monitoring signals. After the analysis module 421 finishes the analysis, the analysis module 451 starts analyzing the physiological monitoring signals. In some embodiments, the analysis modules operate simultaneously.

The transmission unit 430 successively outputs the analysis results S_{A1} and S_{A2} to the external device. In one embodiment, all external devices receive the analysis results S_{A1} and S_{A2}. Therefore, a user can watch different physiological symptoms in a single external device. In other embodiments, some external devices receive the analysis result S_{A1} or S_{A2}.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

While the invention has been described by way of example and in terms of the preferred embodiments, it is to be understood that the invention is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A processing device, comprising:
an interface unit configured to receive a plurality of physiological monitoring signals;
a first slot configured to hold a first analysis module, wherein when the first analysis module is inserted into the first slot, the first analysis module analyzes a first signal group to generate a first analysis result, and the first signal group comprises at least two of the physiological monitoring signals; and
a transmission unit outputting the first analysis result to at least one external device.

2. The processing device as claimed in claim 1, wherein when a second analysis module is inserted into the first slot, the second analysis module analyzes a second signal group to generate a second analysis result different from the first analysis result, and the second signal group comprises at least two of the physiological monitoring signals.

3. The processing device as claimed in claim 2, wherein at least one physiological monitoring signal among the first signal group is the same as at least one physiological monitoring signal among the second signal group.

4. The processing device as claimed in claim 1, further comprising:
a second slot configured to hold a second analysis module, wherein when the second analysis module is inserted into the second slot, the second analysis module analyzes a second signal group to generate a second analysis result, and the second signal group comprises at least two of the physiological monitoring signals.

5. The processing device as claimed in claim 4, wherein at least one physiological monitoring signal among the first signal group is the same as at least one physiological monitoring signal among the second signal group.

6. The processing device as claimed in claim 4, wherein the transmission unit outputs the first analysis result to a first external device and outputs the second analysis result to a second external device.

7. The processing device as claimed in claim 4, wherein the transmission unit outputs the first and second analysis results to the external device.

8. The processing device as claimed in claim 1, wherein the external device displays the first analysis result as text or an image.

9. The processing device as claimed in claim 1, wherein the first analysis module comprises a analysis unit to analyzes the first signal group to generate an output signal.

10. The processing device as claimed in claim 9, wherein the transmission unit serves the output signal as the first analysis result and outputs the output signal to the external device, and the external device further comprises a report unit collects the output signal to generate a report result.

11. The processing device as claimed in claim 9, wherein the external device is a storage device storing the report result.

12. The processing device as claimed in claim 9, wherein the external device is a display device displaying the report result as text or an image.

13. The processing device as claimed in claim 9, wherein the first analysis module further comprises a report unit collecting the output signal to generate the first analysis result.

14. The processing device as claimed in claim 13, wherein the first analysis module further comprises a control unit, and when a state of the first signal group conforms to a danger state, the control unit sends an alarm signal.

15. The processing device as claimed in claim 1, wherein the transmission unit outputs the first analysis result according to a wired transmission technology or a wireless transmission technology.

16. The processing device as claimed in claim 1, wherein the physiological monitoring signals comprise at least two of an electroencephalography (EEG) signal, a phonocardiograph (PCG) signal, an impedance cardiogram (ICG) signal, a photoplethysmogram (PPG) signal, a blood pressure signal, an electrocardiogram (ECG) signal, a respiration signal, an electromyography (EMG) signal and a heart rate signal.
